(19) United States
(12) Patent Application Publication
(10) Pub. No.: US 2025/0049412 A1
(43) Pub. Date: Feb. 13, 2025

(54) REGISTRATION PHANTOM

(71) Applicants: Siemens Medical Solutions USA, Inc., Malvern, PA (US); Siemens Healthineers AG, Forchheim (DE)

(72) Inventors: Dieter RITTER, Fuerth (DE); Martin ZIGANN, Erlangen (DE); Paul SCHLEYER, Knoxville, TN (US)

(73) Assignees: Siemens Medical Solutions USA, Inc., Malvern, PA (US); Siemens Healthineers AG, Forchheim (DE)

(21) Appl. No.: 18/793,099

(22) Filed: Aug. 2, 2024

Related U.S. Application Data
(60) Provisional application No. 63/532,151, filed on Aug. 11, 2023.

(51) Int. Cl. A61B 6/58; A61B 6/00; A61B 90/00; G01R 33/58

(52) U.S. Cl. CPC A61B 6/584; A61B 6/5247; G01R 33/58; A61B 2090/392; A61B 2090/3954

(57) ABSTRACT

A phantom is suitable for registration of a plurality of modalities of a multimodality imaging system. The phantom comprises a plurality of markers, which are embedded in a holding structure. The plurality of markers can be placed in a vertical drilling of the holding structure. Furthermore, the plurality of markers may be arranged in the form of a helix. The markers may be placed in drillings of the holding structure, wherein the direction of the drillings is perpendicular to the axis of the helix.

### CROSS-REFERENCE TO RELATED APPLICATION(S)

The present application claims priority under 35 U.S.C. § 119(e) to U.S. Application No. 63/532,151, filed Aug. 11, 2023, the entire contents of which is incorporated herein by reference.

### BACKGROUND

Independent of the grammatical term usage, individuals with male, female or other gender identities are included within the term.

Multimodality imaging plays an important role in accurately identifying diseased and normal tissues. Multimodality imaging provides combined benefits by fusing images acquired by different modalities. The complementarity between anatomic (e.g., computed tomography (CT) and magnetic resonance (MR) imaging) and molecular (e.g., positron-emission tomography (PET)) imaging modalities, for instance, has led to the widespread use of PET/CT and PET/MR imaging.

Multimodality scanners require a procedure to measure the spatial displacement between images produced by the different modalities (e.g., PET to CT displacement, or PET to MR displacement). This procedure is commonly referred to as “gantry alignment”. The standard gantry alignment procedure uses radioactive sources or hot phantoms that facilitate the acquisition of PET emission data to form the PET image. Radioactive sources (e.g. points sources, line sources) are typically positioned in a specific arrangement and imaged using, for example, both PET and CT for a PET/CT scanner, or both PET and MR for a PET/MR scanner.

For a PET/CT scanner, the radioactive source material typically produces sufficient X-ray attenuation to produce a CT image. For a PET/MR scanner, the radioactive source material is typically not visible in MR imaging sequences, so the radioactive sources are traditionally surrounded by an MR-visible material, such as oil, which produces an MR image. The MR-invisible radioactive source material results in a void in the MR images, which is used to identify the location of the sources.

However, such gantry alignment procedure that relies on an external radioactive positron source is typically time consuming, since the source must be maintained and carefully placed in the center of the gantry of the scanner. Additionally, a human needs to handle the source repeatedly, which leads to health and safety risks due to the radioactivity of the source.

US 2022/0398754 A1 (which is hereby incorporated by reference) describes a framework for gantry alignment of a multimodality medical scanner. First image data of a non-radioactive structure is acquired by using intrinsic radiation emitted by scintillator crystals of detectors in a first gantry of the multimodality medical scanner. Second image data of the non-radioactive structure is acquired using a second gantry for another modality of the multimodality medical scanner. Image reconstruction may be performed based on the first and second image data of the non-radioactive structure to generate first and second reconstructed image volumes. A gantry alignment transformation that aligns the first and second reconstructed image volumes may then be determined.

### SUMMARY

It is an objective of one or more embodiments of the present invention to provide a phantom which is particularly suitable for registration of two modalities of a multimodality imaging system.

An embodiment of the present invention provides a phantom for registration of a plurality of modalities of a multimodality imaging system, the phantom comprising: a plurality of markers, which are embedded in a holding structure.

According to one or more embodiments, the plurality of markers may exhibit a greater visibility than the holding structure in at least one of MR or CT images. The holding structure may be invisible in MR images.

The plurality of markers may include a solid material.

The plurality of markers may comprise or consist of an elastomer material.

The plurality of markers may include a material that exhibits a single-peak frequency response in an MR-measurement.

The plurality of markers may lack ferromagnetic material additives.

The plurality of markers may have one of a spherical shape, a u-shape or an x-shape.

The spherical shape may have a diameter of less than or equal to 50 mm, less than or equal to 40 mm or less than or equal to 30 mm.

The plurality of markers may be made of a material that shows a level of attenuation with regard to the holding structure sufficient to identify the plurality of markers using intrinsic radiation emitted by scintillator crystals of PET detectors of a multimodal imaging system.

The plurality of markers may be distributed inside the holding structure according to a weighted random spatial distribution.

The holding structure may comprise or consist of a foam material.

The phantom may further include a mounting structure on which the holding structure is mounted.

The plurality of markers may be placed in a vertical drilling of the holding structure.

The holding structure may comprise cavities to receive the plurality of markers, wherein dimensions of the cavities match dimensions of the plurality of markers.

The plurality of markers may be arranged in the form of a helix, such as a double entwined helix.

The plurality of markers may be placed in drillings of the holding structure, and a direction of the drillings may be perpendicular to an axis of the helix.

The plurality of markers may be arranged a same distance from a central point.

The distance from the central point may be between 70 mm and 150 mm, between 90 mm and 130 mm or between 105 mm and 115 mm.

The plurality of markers may be uniformly distributed along an axis of the phantom.

The plurality of markers may be placed in drillings of the holding structure, and a direction of the drillings may be perpendicular to the axis of the phantom.

Another embodiment of the present invention provides a multimodality imaging system, comprising a phantom for registration of a plurality of modalities of a multimodality imaging system. The phantom includes a plurality of markers, which are embedded in a holding structure.

The phantom may further include a mounting structure on which the holding structure is mounted, wherein the mounting structure is fixed to the multimodality imaging system such that the holding structure is placeable within the isocenter of the multimodality imaging system.

The multimodality imaging system may further include a patient table, and the phantom may include a mounting structure on which the holding structure is mounted, the mounting structure being fixed to the patient table.

The mounting structure may be fixed to the patient table such that the holding structure is placed beyond a head end of the patient table.

### BRIEF DESCRIPTION OF THE DRAWINGS

The present invention will be described and explained in greater detail below on the basis of the example embodiments shown in the figures:

FIG. 1 shows a first embodiment of the phantom.

FIG. 2 shows a second embodiment of the phantom.

FIG. 3 shows an MR image of an embodiment of the phantom.

FIG. 4 shows a helical arrangement of the markers according to an embodiment of the phantom.

FIG. 5 shows an embodiment of the phantom comprising a mounting structure.

FIG. 6 shows an embodiment of the multimodality imaging systems comprising an embodiment of the phantom.

FIGS. 7 and 8 show a registration of the centers of spherical markers of an embodiment of the phantom in order to align two modalities of an imaging system.

FIG. 9 shows the MR frequency response spectrum of a marker according to an embodiment of the phantom.

### DETAILED DESCRIPTION

Specific structural and functional details disclosed herein are merely representative for purposes of describing example embodiments of the present invention. This invention may, however, be embodied in many alternate forms and should not be construed as limited to only the embodiments set forth herein.

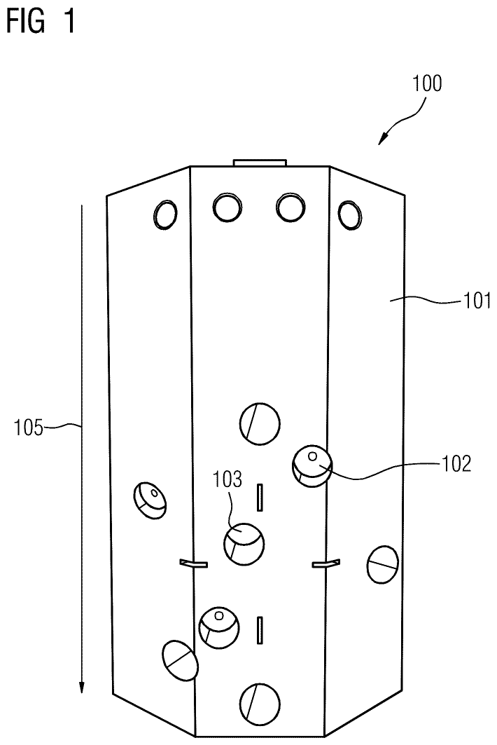

FIG. 1 shows a first embodiment of the phantom (100) for registration of two modalities of a multimodality imaging system. The phantom (100) comprises a plurality of markers (102), which are embedded in an holding structure (101). The markers (102) exhibit a greater visibility in MR and/or CT images than the holding structure (101). Preferably, the holding structure (101) is invisible in MR images.

The holding structure (101) consists of foam material. One material of choice for the holding structure (101) is a hard foam. The material for the holding structure (101) should be chosen in a way so that the holding structure (101) has low visibility in PET and CT examinations. Nevertheless, the holding structure (101) should have enough stability so that the markers (102) can be placed at designated volumetric positions. Furthermore, the placement of the markers (102) within the holding structure (101) should be possible without complex mechanical mounting. All in all, the holding structure (101) should support precise feature extraction of the embedded geometry of the markers (102).

The phantom (100) as depicted in FIG. 1 has the shape of a prism, in particular a prism with two octagonal bases. Of course, any other suitable shape can be contemplated for the phantom (100), e.g. a cylindrical shape.

The markers (102) are placed in vertical drillings (103) of the holding structure (101). This allows easy manufacturing of the phantom (100). In addition, the holding structure (101) comprises cavities in which the markers (102) are placed, wherein the dimensions of the cavities each match the dimensions of the markers (102). After insertion of the markers (102) into the vertical drillings (103), the openings of the vertical drillings (103) can be closed by filling the openings with plugs. Such plugs max fix the markers (102) at the appropriate locations.

The holding structure (101) according to FIG. 1 has a symmetry axis (105). The plurality of markers (102) are uniformly distributed along this axis (105). The uniform distribution prevents or reduces x-ray or gamma-ray shadowing in the transversal direction.

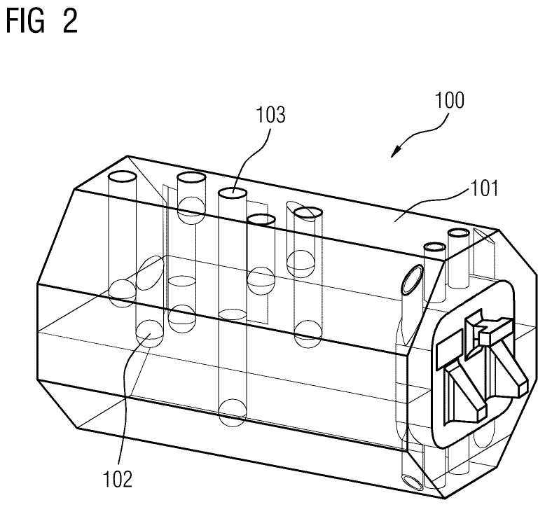

FIG. 2 shows a second embodiment of the phantom (100) and provides a detailed view how the markers (102) can be placed inside the vertical drillings.

In particular, FIG. 2 shows that the direction of the drillings (103), in which the markers (102) are placed, is perpendicular to the axis (105) of the phantom (100). I.e. the vertical drillings (103) are directed parallel to the octagonal bases of the prism. In case the phantom (100) has the shape of a cylinder, the vertical drillings (103) are preferably directed parallel to the base circles of the cylinder. As shown in FIG. 1, the vertical drillings (103) have different depths so that the markers (102) are placed with varying distance to the surface of the phantom.

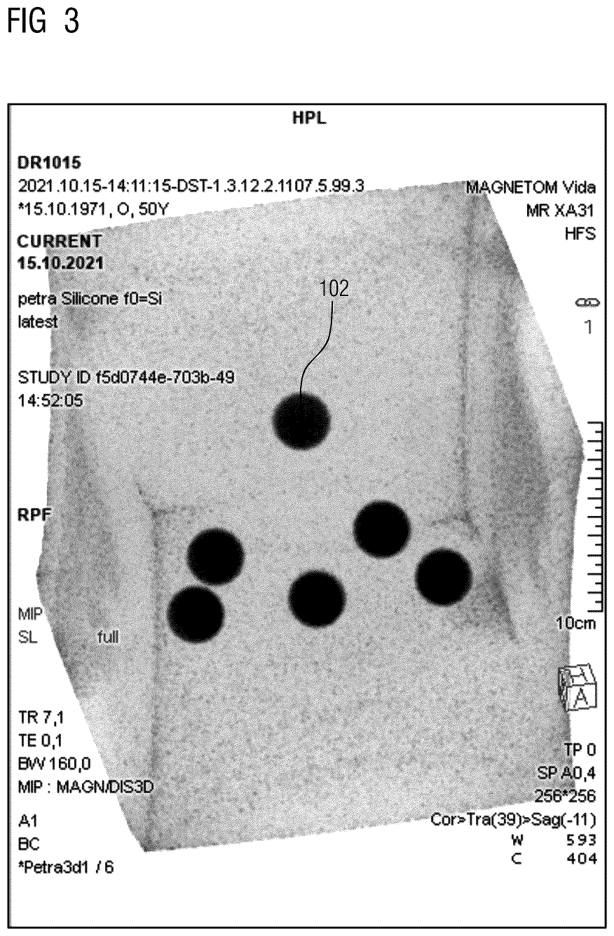

FIG. 3 shows an MR image of an embodiment of the phantom (100). As can be conceived by FIG. 3, the markers (102) are made of a material, which is MR-visible. In particular, the markers (102) consist of a solid material. Such material should be durable. This prevents diffusion and/or evaporation of liquid material, which would lead to air bubbles which would affect the precise determination of the shape of the markers (102).

Preferably, the markers (102) consist of an elastomer material. Elastomer materials can e.g. comprise silicon material, rubber material or plastic material. Preferably, industrial rather than natural elastomers are used as a material for the markers (102). This allows better reproducibility due to constant material parameters. Furthermore, the stability of the material can be improved due to e.g. drying and hardening over time. The material should have a reasonable hardness grade and show enough stability and density. The markers (102) should be geometrically stable and should not deform under their own weight.

The choice of material for the markers (102) allows sufficient proton signal and therefore sufficient volume signal-to-noise ratio (SNR) for MR imaging and sufficient local SNR for MR adjustment measurements. Furthermore, the material of the markers (102) should be chosen such that the markers (102) show sufficient attenuation of the PET and/or CT photons.

Furthermore, the markers (102) lack ferromagnetic material additives, e.g. color particles. It should be ensured that the markers (102) are not contaminated with such ferromagnetic materials.

The markers (102) should be made of a material which shows enough attenuation with regard to the holding structure, so that the markers (102) can be identified in PET and/or CT imaging. In particular, the markers (102) should be made of a material which shows enough attenuation with regard to the holding structure, so that the markers (102) can be identified by using the intrinsic radiation emitted by scintillator crystals of PET detectors of the multimodality imaging system. Therefore, a suitable material should be chosen for the markers (102) so that identification of the markers based on the background radiation of the PET detectors, in particular the background radiation of the lutetium orthosilicate (LSO) or lutetium yttrium orthosilicate (LYSO) scintillator crystals, is possible.

Furthermore, the markers (102) shown in FIG. 3 have a spherical shape. Other shapes, like a u-shape or an x-shape, can also be contemplated. The diameter of the spherical shape is at most 50 mm, preferably at most 40 mm, preferably at most 30 mm. This shape and/or size of the markers (102) allows a stable and precise feature extraction. The markers (102) are distributed inside the holding structure (101) according to a weighted random spatial distribution. Any other spatial distribution can be contemplated for the markers (102).

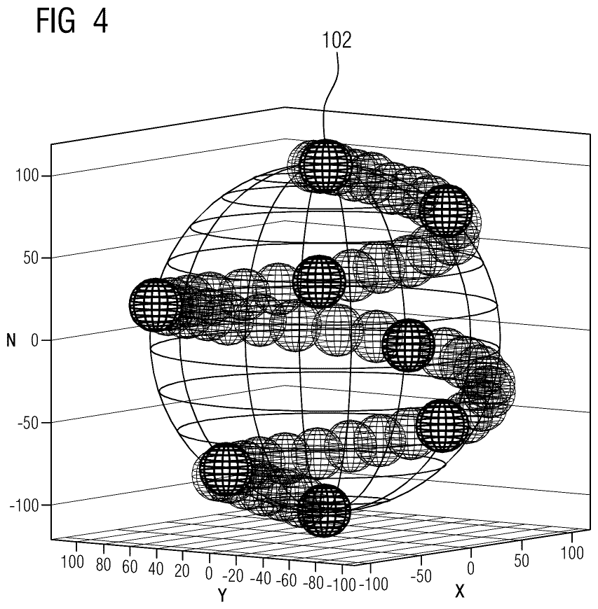

FIG. 4 shows a helical arrangement of the markers (102) according to an embodiment of the phantom (100), i.e. the plurality of markers (102) are arranged in form of a helix. As shown in FIG. 4 the markers (102) are arranged in form of a double entwined helix.

Such helical placement of the markers (102) allows a homogeneous distribution of the markers (102) in 3D space. The markers (102) are distributed equally along the transversal 720 degree rotation in order to enable a good coverage of the volume. Furthermore, a 60 degree transversal offset is used so that the drilling is simplified in a direction perpendicular to the axis of the helix. Furthermore, by using this offset the markers (102) can be placed in different depths into the foam of the holding structure (101).

Furthermore, the helical placement of the markers (102) can minimize the overlap of the markers (102) in case an orthogonal projection over an 3D image taken from the phantom (100) is performed. I.e. due to the helical placement of the markers (102) it can be avoided that in an orthogonal projection a first marker (102) is hidden behind a second marker (102) leading. Therefore, the helical placement of the markers (102) allows clear identification of the markers (102) in orthogonal projections. This can lead to more accurate 2D registrations of orthogonal projections of images of the phantom (100) acquired by two modalities of an imaging system, wherein such 2D registrations are performed to align the two modalities of the imaging system.

In particular, the markers (102) the direction of the drillings (103) shown in FIG. 1 is perpendicular to the axis of the helix.

Furthermore, as shown in FIG. 4 the markers (102) are arranged with the same distance from a central point. The distance from the central point is between 70 and 150 mm, preferably between 90 mm and 130 mm, preferably between 105 mm and 115 mm. Thereby, the markers (102) are placed on the surface of a sphere having a diameter of e.g. 210 mm. In this example, a homogeneous spherical volume with a 240 mm diameter can be filled with the markers (102) leading to a good tradeoff between geometric accuracy and magnetic field distortion.

The number of markers (102) contained by the phantom (100) can range between 3 and 15 markers (102), preferably between 5 and 12 markers (102), preferably between 7 and 10 markers (102). Preferably 8 markers (102) are embedded in the phantom (100).

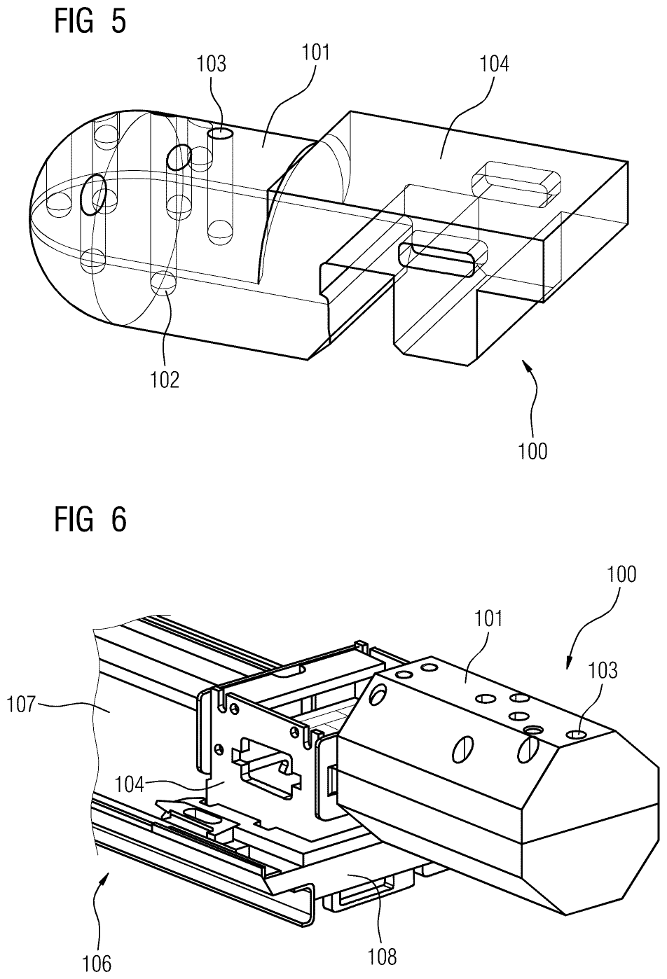

FIG. 5 shows an embodiment of the phantom (100) comprising a mounting structure (104). The holding structure (101) is mounted on the mounting structure (104).

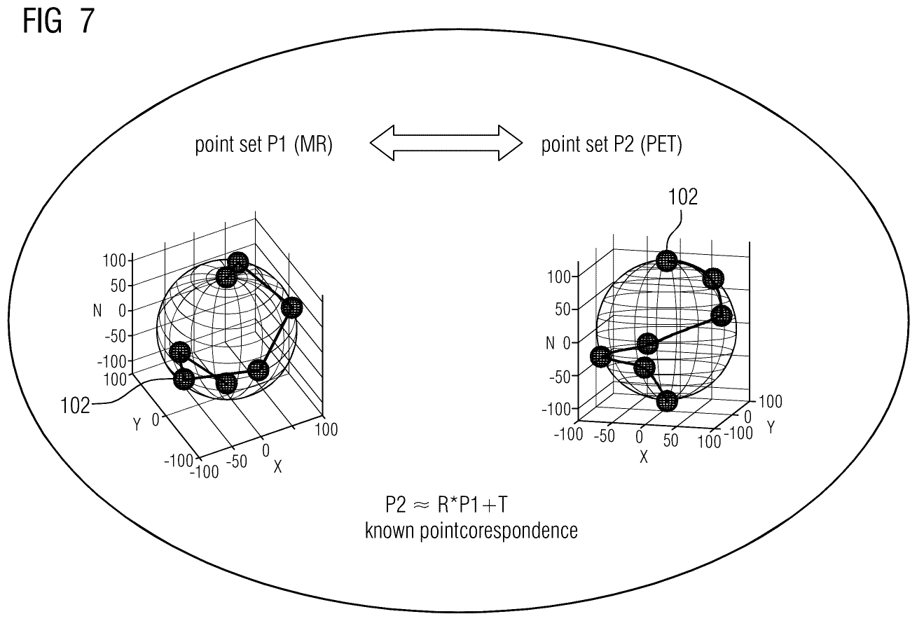

FIG. 6 shows an embodiment of the multimodality imaging systems (106) comprising an embodiment of the phantom (100).

The multimodality imaging system (106) is preferably a combination of a magnetic resonance (MR) imaging system and an emission tomography imaging system. The emission tomography imaging system is preferably a positron emission tomography (PET) imaging system or a single photon emission tomography (SPECT) imaging system.

The phantom (100) comprises a mounting structure (104) on which the holding structure (101) is mounted. The mounting structure (104) is fixed to the multimodality imaging system (106) in a way so that the holding structure (101) is placeable within the isocenter of the multimodality imaging system (106). This allows a placement of the markers (102) within the homogeneous imaging volume of the multimodality imaging system (106).

In FIG. 6 only a part of the multimodality imaging system (106), namely the patient table (107) of the multimodality imaging system (106), is shown. Other parts of the multimodality imaging system (106) can be easily contemplated by the skilled person.

The mounting structure (104) is fixed to the patient table (107). Thereby, the holding structure (101) is fixed in a defined position to the patient table (107). The mounting structure (104) is fixed to the patient table (107) in a way so that the holding structure (101) is placed beyond a head end (108) of the patient table (107). This allows avoiding or reducing x-ray or gamma-ray attenuation which is induced by the phantom (100).

Preferably, the helical arrangement of the markers (102) as shown in FIG. 4 is used in combination with the mounting of the phantom (100) as shown in FIG. 5. Therefore, the holding structure (101) can be placed in head-feet direction of the patient table (107) a certain distance away from the head end (108) of the patient table (107). E.g. such distance is at least 90 mm, preferably at least 120 mm, preferably at least 150 mm, preferably at least 180 mm. Thereby, B0-field influences of eddy currents of screws in the holding structure (101) can be reduced on the MR imaging volume (leading to reduced geometric image distortion). As an example the 240 mm diameter volume of the holding structure (101) which is homogeneously filled with the markers (102) can be placed 180 mm away (in head direction) from any standard phantom holder.

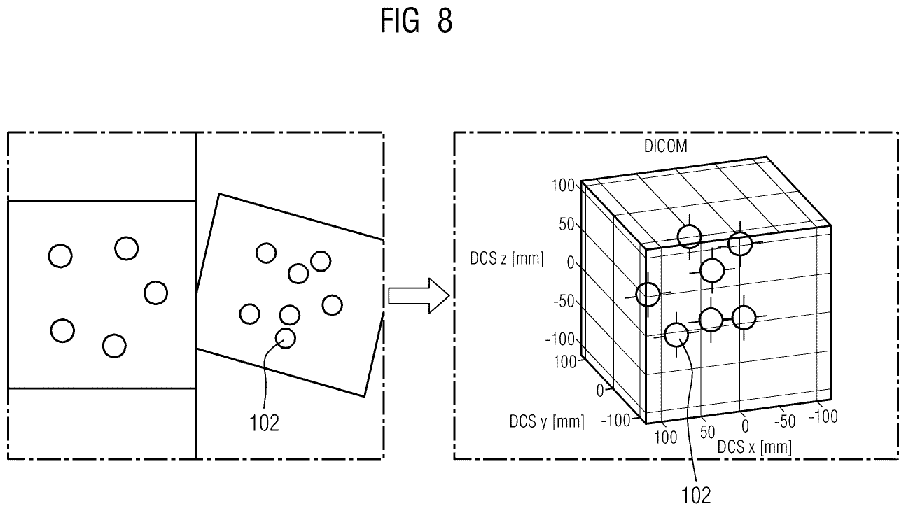

FIGS. 7 and 8 show a registration of the centers of spherical markers (102) of an embodiment of the phantom (100) in order to align two modalities of an imaging system.

This registration can be seen as a part of the image registration described in US 2022/0398754. Thereby, the phantom (100), especially the markers (102) of the phantom (100), can be seen as the non-radioactive structure positioned in a field-of-view of a multimodality imaging system (106) described in US 2022/0398754.

First image data of the phantom (100) are acquired by using intrinsic radiation emitted by scintillator crystals of positron-emission tomography (PET) detectors in the multimodality imaging system (106).

Second image data of the phantom (100) are acquired using magnetic resonance (MR) or computed tomography (CT) of the multimodality imaging system (106).

If using MR imaging as the second image modality, the use of special MR pulse sequences can be contemplated in order to increase the visibility of the markers (102) in the acquired MR-images. E.g. MR pulse sequences with a short echo time, e.g. shorter than 1 ms, can be used. It is also possible to 3D volume MR pulse sequences.

E.g. the use of a fl3d_ce pulse sequence can be contemplated to acquire the MR imaging data with following parameters:
Imaging volume: 240 mm×240 mm×240 mm
Resolution: 0.9 mm isotrop
FOV=240 mm
ST=0.94 mm
Res=256
DisCorr=Dis3D
α=25° slabs=1
slices=256
TR=20 ms
Option=minTE
Excitation=non-selective
RFpulse=fast
GradMode=boost
Pbw=1150 Hz/pixel
TE=0.71 ms
Switch off GSWD
   E.g. the use of a fl3d_ce pulse sequence can be contemplated to acquire the MR imaging data with following parameters:
Imaging volume: 240 mm×240 mm×240 mm
Resolution: 0.9 mm isotrop
FOV=240 mm
Res=256
DisCorr=Dis3D
α=6°
TR=5 ms
RadViews=25000
Pbw=240 Hz/pixel
TE=0.07 ms

Image reconstruction is performed based on the first and second image data of the phantom (100) to generate first and second reconstructed image volumes. A gantry alignment transformation that aligns the first and second reconstructed image volumes is determined.

The gantry alignment transformation is based on the detected markers (102), e.g. the centers of the spherical markers (102). Such markers (102) are detected, preferably with subpixel precision, in the first and second image data, i.e. in the two corresponding 3D image data sets (PET/MR or PET/CT).

For gantry alignment, a rigid 3D registration, e.g. in a least square sense, can be used. FIG. 7 shows the schematics of the rigid 3D registration in a least square sense of the MR point set and the PET point set. The registration can be based on intensity and mutual information. Least square sense matching of projections of both 3D data sets onto orthogonal planes is also possible. All registration procedures can come with an accuracy estimation for report, if required. FIG. 8 shows a Hough transform between the subvoxel center coordinates.

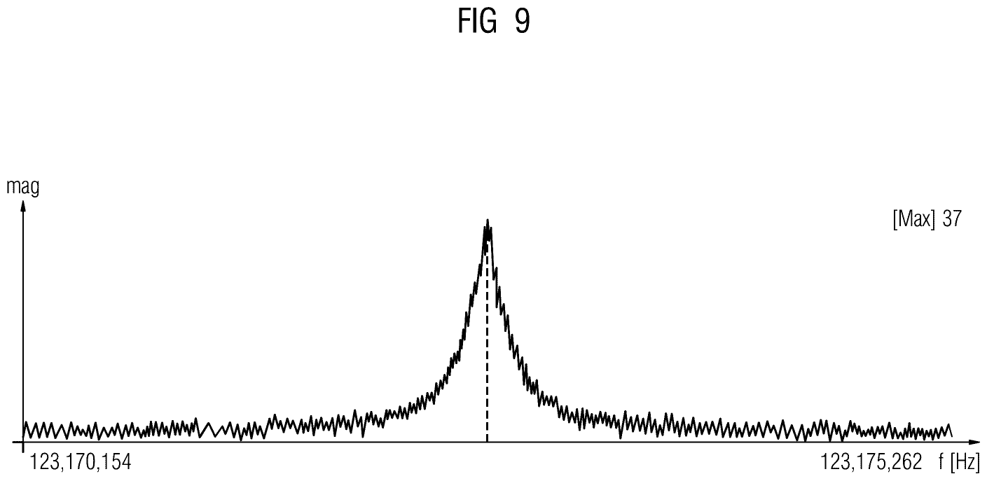

FIG. 9 shows the MR frequency response spectrum of a marker (102) according to an embodiment of the phantom. (100). The markers (102) consist of a material which exhibits a single-peak frequency response in the MR-measurement, e.g. an MR adjustment measurement (frequency adjustment and/or transmitter adjustment).

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A phantom for registration of a plurality of modalities of a multimodality imaging system, the phantom comprising:
a plurality of markers, which are embedded in a holding structure.

2. The phantom according to claim 1, wherein the plurality of markers exhibit a greater visibility than the holding structure in at least one of MR or CT images.

3. The phantom according to claim 1, wherein the holding structure is invisible in MR images.

4. The phantom according to claim 1, wherein the plurality of markers include a solid material.

5. The phantom according to claim 1, wherein the plurality of markers comprise an elastomer material.

6. The phantom according to claim 5, wherein the plurality of markers consist of the elastomer material.

7. The phantom according to claim 1, wherein the plurality of markers include a material that exhibits a single-peak frequency response in an MR-measurement.

8. The phantom according to claim 1, wherein the plurality of markers lack ferromagnetic material additives.

9. The phantom according to claim 1, wherein the plurality of markers have one of a spherical shape, a u-shape or an x-shape.

10. The phantom according to claim 9, wherein the plurality of markers have a spherical shape with a diameter of less than or equal to 50 mm.

11. The phantom according to claim 1, wherein the plurality of markers are made of a material that shows a level of attenuation with regard to the holding structure sufficient to identify the plurality of markers using intrinsic radiation emitted by scintillator crystals of PET detectors of a multimodal imaging system.

12. The phantom according to claim 1, wherein the plurality of markers are distributed inside the holding structure according to a weighted random spatial distribution.

13. The phantom according to claim 1, wherein the holding structure comprises a foam material.

14. The phantom according to claim 13, wherein the holding structure consists of the foam material.

15. The phantom according to claim 1, further comprising:
a mounting structure on which the holding structure is mounted.

16. The phantom according to claim 1, wherein the plurality of markers are placed in a vertical drilling of the holding structure.

17. The phantom according to claim 1, wherein the holding structure comprises cavities to receive the plurality of markers, and wherein dimensions of the cavities match dimensions of the plurality of markers.

18. The phantom according to claim 1, wherein the plurality of markers are arranged in the form of a helix.

19. The phantom according to claim 18, wherein the plurality of markers are arranged in the form of a double entwined helix.

20. The phantom according to claim 18, wherein
the plurality of markers are placed in drillings of the holding structure, and
a direction of the drillings is perpendicular to an axis of the helix.

21. The phantom according to claim 1, wherein the plurality of markers are arranged a same distance from a central point.

22. The phantom according to claim 21, wherein the distance from the central point is between 70 mm and 150 mm.

23. The phantom according to claim 1, wherein the plurality of markers are uniformly distributed along an axis of the phantom.

24. The phantom according to claim 23, wherein
the plurality of markers are placed in drillings of the holding structure, and
a direction of the drillings is perpendicular to the axis of the phantom.

25. A multimodality imaging system, comprising a phantom according to claim 1.

26. The multimodality imaging system according to claim 25, wherein the phantom comprises:
a mounting structure on which the holding structure is mounted, wherein
the mounting structure is fixed to the multimodality imaging system such that the holding structure is placeable within the isocenter of the multimodality imaging system.

27. The multimodality imaging system according to claim 25, further comprising:
a patient table; and wherein
the phantom includes a mounting structure on which the holding structure is mounted, the mounting structure being fixed to the patient table.

28. The multimodality imaging system according to claim 27, wherein the mounting structure is fixed to the patient table such that the holding structure is placed beyond a head end of the patient table.

29. The phantom according to claim 9, wherein the plurality of markers have a spherical shape with a diameter of less than or equal to 40 mm.

30. The phantom according to claim 9, wherein the plurality of markers have a spherical shape with a diameter of less than or equal to 30 mm.

31. The phantom according to claim 21, wherein the distance from the central point is between 90 mm and 130 mm.

32. The phantom according to claim 21, wherein the distance from the central point is between 105 mm and 115 mm.
